(19) United States
(12) Patent Application Publication
(10) Pub. No.: US 2024/0002769 A1
(43) Pub. Date: Jan. 4, 2024

(54) STRUCTURED MONOLITHIC FIXED BED FOR CELL CULTURE, RELATED BIOREACTOR AND METHODS OF MANUFACTURING

(71) Applicant: UNIVERCELLS TECHNOLOGIES S.A., Nivelles (BE)

(72) Inventors: Christophe Dumont, Nivelles (BE); Tania Pereira Chilima, Nivelles (BE); Antoine Hubert, Nivelles (BE); Jean-Christophe Drugmand, Wavre (BE); José Castillo, Brussels (BE); Sebastien Rodriguez, Nivelles (BE); Bastien Mairesse, Nivelles (BE)

(21) Appl. No.: 18/039,879

(22) PCT Filed: Dec. 2, 2021
(86) PCT No.: PCT/EP2021/084024

§ 371 Date: Jun. 1, 2023

Related U.S. Application Data
(60) Provisional application No. 63/153,082, filed on Feb. 24, 2021.

(30) Foreign Application Priority Data
Dec. 2, 2020 (WO) PCT/EP2020/084317

(51) Int. Cl. C12M 1/12

(52) U.S. Cl. CPC C12M 25/18; C12M 25/14

(57) ABSTRACT

An apparatus for culturing cells includes a bioreactor including a monolithic structured cell culture bed, which may be fabricated using additive manufacturing techniques, such as for example 3D printing. The bioreactor and monolithic structured cell culture bed may be formed as a unitary structure. The monolithic structured cell culture bed may include regions of varying porosity and/or surface treatments to achieve desired objectives. Related methods are also provided.

This application claims the benefit of U.S. Provisional Patent Application Ser. No. 63/153,082, filed Feb. 24, 2021, the disclosure of which is incorporated by reference. This application further claims the benefit of international patent application Ser. No. PCT/EP2020/084317, filed Dec. 2, 2020, the disclosure of which is incorporated by reference.

This application is related to U.S. Provisional Patent Application Ser. No. 62/942,345, filed Dec. 2, 2019, and U.S. Provisional Patent Application Ser. No. 63/004,706, filed Apr. 3, 2020, the disclosures of which are incorporated herein by reference. This application further incorporates by reference the following applications: U.S. Provisional Patent Application Ser. Nos. 62/758,152, 62/733,375, and 62/608,261; U.S. Patent Application Publication No. 2018/0282678; International Patent Application PCT/EP2018/076354; U.S. Provisional Patent Application 62/711,070; and U.S. Provisional Patent Application 62/725,545.

### TECHNICAL FIELD

This document relates generally to the cell culturing arts and, more particularly, to a structured monolithic fixed bed for cell culturing, a related bioreactor, and methods of manufacturing.

### BACKGROUND

Bioreactors are sometimes used for growing (or culturing) certain cells within cell culture beds, which may be fixed in place within the bioreactor. Such a fixed bed may be unstructured (e.g., formed of loose particles packed together) or may be structured in some manner. Such structured fixed beds are often formed of discrete layers of material carefully placed on top of or beside one another. In either case, these unstructured fixed beds carry a significant cost and complexity to manufacture, particularly in terms of the amount of human involvement required. Specifically, the fibers or layers must be manually assembled in a particular manner to achieve a cell culture bed suitable for given process conditions.

Oftentimes, homogeneity of fluid flow and cell growth may be lacking unless special care is taken during fabrication of the bed. Specifically, cell viability and growth may also be compromised as a result of flow restrictions within the bed caused by variations in density, which may result from unpredictable variations in the materials used. Such variations can create high pressure gradients in some areas where fluid tends not to readily flow.

Likewise, it is sometimes desirable to vary the fluid flow within a cell culture bed. To achieve this, complex arrangements have been proposed using different types of materials or special geometries, again with involved fabrication techniques that lead to increased cost and complexity. In situations where the grown cells are the target to be harvested, maximizing the recovery may also be challenging using existing fixed bed arrangements, which are of generally dense and not well-suited or optimized for achieving viable cell detachment and maximizing recovery. In addition, loosened fibers and friction-created particles may be produced and freed from these traditional fixed beds due to agitation, fluid pressure or other forces applied to the bioreactor. For instance, particles may be created and freed from the bed when adjacent layers of material rub against one another during the process of culture and harvest.

Accordingly, a need is identified for a structured fixed bed that overcomes the foregoing issues and perhaps others yet to be discovered. Specifically, there is a desire to provide a structured fixed bed for a bioreactor with a customizable structure that does not rely on manual assembly to create, and thus can reduce costs. The customizable nature would allow for the creation of a fixed bed with different flow characteristics in different regions in a highly predictable and repeatable manner. The structured fixed bed could eliminate the interlayer friction that could cause particles that would contaminate the harvest. The structured fixed bed could also be concurrently formed as part of the bioreactor, thus eliminating the need for separate fabrication steps, and could be designed as a compressible structure to enhance fluid recovery and cell harvesting.

### SUMMARY

In one aspect, this disclosure pertains to a structured fixed bed comprising a three-dimensional (3D) monolith, such as in the form of a scaffold or lattice formed of multiple interconnected units or objects. Such objects have surfaces for cell adhesion. The fixed bed may be single use in nature to avoid the cost and complexities involved in cleaning according to bioprocessing standards. Such a monolithic structured fixed bed would prevent the generation of particles (fixed-bed containing PET fibers could release some free fibers), which allows for use in processes for which the product can be filtered at the end (e.g. stem cells applications of production of large viruses than cannot be sterile filtered).

In a further aspect, the present disclosure is directed to an apparatus comprising a cell culture vessel (e.g., a bioreactor) and the monolithic structured fixed bed. In one further embodiment, the structured fixed bed comprises a large-scale cell matrix for high-density adherent cell growth in a cell culture system.

In one embodiment, the fixed bed is manufactured using additive manufacturing, such as for example 3-D printing technology. In some embodiments, the fixed bed is manufactured using selective laser sintering (SLS) 3D printing, which employs a high-powered laser to fuse powdered material together into a desired 3D shape. However, other techniques may also be used, such as for example stereolithography or “SLA,” Fused Deposition Modeling (FDM), Digital Light Process (DLP), Multi Jet Fusion (MJF), PolyJet, Direct Metal Laser Sintering (DMLS), or Electron Beam Melting (EBM) [symbol] he fixed bed may comprise one or more objects that are fused or sintered together to form a monolithic matrix, which may comprise one or more structures. The matrix may provide one or more linear or non-linear (e.g., tortuous) paths for fluid and cells to flow therethrough when in use.

In some embodiments, the object may comprise one or more shapes. The one or more objects may include, but not be limited to, shapes such as spherical, oval, elliptical, cubic, pyramidal, hexagonal, octagonal, decahedral, square, rectangular or any combinations of the foregoing, and may be created from a repetition of simple shapes. The objects may be in the form of beads or other small simple shapes (which may or may not be spherical), and may be bonded together, such as a result of the 3D printing process. While other shapes may be used to manufacture the fixed bed structure, pre-positioning of the objects may be done to achieve a homogeneous structure and thus, a simple sphere or ball is desired in this arrangement due to the lack of any need to position before bonding, but any other shapes could be used

The objects may be bound, printed or fused together to form the fixed bed structure or matrix. The methods of bonding or fusing can include local welding, SLS or other 3D printing methodologies, but are not limited to such techniques. The surface area of the fixed bed is equal to the surface area of all of the objects and adherent cells would adhere to such objects in a two-dimensional model or manner.

In some embodiments, the objects are solid and manufactured of polymer material. The objects can alternatively be hollow with a cavity inside. The surface of the objects may be continuous, or could be discontinuous to provide access to the inner cavity inside the object (porosity). The discontinuous portions may form openings that provide access to the cavity and the openings provide a path for cell culture medium to flow therethrough. The openings may be smaller than the size of an average cell, thereby preventing cells from entering the cavity and becoming entrapped inside, but could also be larger to let the cells colonize the cavity. Alternatively, the cavity could be larger to permit cells to enter and grow therein. In any case, the opening(s) can permit cell culture medium to flow therethrough. The fluid flow path through the object may be regular or irregular (uneven). The objects could have several sizes of cavity/porosity as the veins and arteries of the blood circulation. The concept is to mimic design of the blood circulation with cells that could be trapped inside cavities and media irrigated by large channels To get the cells out easily, they may also adhere to the object surface and not be trapped inside a cavity.

In some embodiments, the fixed bed may be integrated into a cell culture vessel or bioreactor. In some embodiments, the structure of the fixed bed is flexible and/or compressible. In some embodiments, the structure comprises one or more monolithic structures integrated in a cell culture vessel or bioreactor. In some embodiments, the matrix and the bioreactor are both manufactured using the same process (e.g., SLS), such that the vessel and the fixed bed are co concurrently formed. This would avoid leakage and bypasses between the matrix and the wall of the vessel, provide for simpler manufacturing process and ensure the right tolerance created between the matrix and the vessel. This would also permit an increased thickness of the vessel wall so that heating occurs at the bottom of the vessel.

The objects that form the fixed bed may be made from a polymer that is compatible in cell culture applications. Suitable materials include, but are not limited to, polystyrene, polyethylene terephthalate, polycarbonate, polyvinylpyrrolidone, polybutadiene, polyvinylchloride, polyethylene oxide, polypyrroles, polypropylene oxide and combinations thereof, or any type of biocompatible polymers that could be used in additive manufacturing technologies. Metals or ceramics could also be used to form the bioreactor, monolithic structured fixed bed, or both.

In some embodiments the fixed bed, objects or a portion thereof may be modified to provide desired cell adhesion properties (i.e., hydrophilization or binding ligands) including treatment processes to modify with various types of plasmas, process gases, and/or chemicals and/or grafting known in the industry. In a preferred embodiment, a surface of the sheets, a monolithic structured fixed bed, or matrix, or a portion thereof is modified or treated.

In some embodiment the objects or fixed bed or a portion thereof may be coated with one or more thin layers of biocompatible hydrogels that may enhance or provide cell adherence properties, including, for example, collagen or Matrigel®. In a further embodiment, the surface to volume ratio of the fixed bed may be between 1 and 10 m² per liter of cell culture medium. In a further embodiment, the fixed bed enables a cell density of greater than 0.1×10⁶ cells/cm² or 10×10⁶ cells/ml of fixed bed.

In some embodiments, the fixed bed is formed to provide maximum homogeneity of fluid flow and cell growth when in use. In some embodiments, the fixed bed is designed to allow efficient and uniform fluid and cell flow therethrough, but could also be designed to guide fluid flow and control fluid dynamics properties, such as pressure, velocity, temperature, or turbulence. In addition, the structure of the fixed bed can accommodate fluid flow in multiple orientations. In some embodiments, the fixed bed comprises sheets that are stacked or folded in a vertical or horizontal orientation. In some embodiments, fluid flows through the sheets vertically and in some embodiments, fluid flows horizontally. The flow of fluid through the sheets may be tortuous. The sheets may be in any shape.

In some embodiments, the monolithic structured fixed bed comprises a shape and size adapted for insertion in a bioreactor. In some embodiments, the monolith is annular. The objects that make up the fixed bed may be identical in size. This results in a fixed bed with homogeneity and the same pressure drop from input to output. However, irregularly or randomly sized or spaced objects may also be used, including for example to create different regions or zones of compaction, porosity or density in portions of the bed in order to create a flow gradient. In essence, the manufacturing of the customizable fixed bed may be such that there are regions or zones of linear fluid flow for feeding the cells and zones of compact structure for entrapping cells (adherent as well as suspension cells) for growth. The characteristics of such bed can be set for different cell types including minimum porosity, surface area, structural zones, linear speed requirements, pressure limitations and needs, etc.

In some embodiments, the fixed bed is manufactured, modified, or adapted to comprise pathways to increase fluid-flow and/or diffusion. These pathways may be formed during manufacturing (for example the 3D printing process) of the fixed bed, or may be formed after the fixed bed is manufactured, such as by using laser or other drilling techniques. In some embodiments, the pathways decrease pressure drops across the fixed bed. For instance, matrices can be stacked one on top of another. In this case, a pressure drop may build up so that a reduction in such pressure drop is needed to maintain homogeneity.

In some embodiments, the fixed bed pathways act to increase fluid flow in both main fluid flow direction and radial diffusion of fluid and cells. Such pathways can follow a convection model, having an irregular (e.g., tortuous or zigzag) path or other (more linear) directional path, with components in a main fluid flow direction as well other components with some direction at an angle toward the perpendicular or radial direction. In some embodiments, the fixed bed comprises one or more vertical, or “chimney,” pathways that increase diffusion and decrease pressure drop across the fixed bed. In some embodiments, the diameter of the pathways is between 1 and 2 mm and the distance between pathways may be from 1 to 4 mm.

In some embodiments, certain portions of the fixed bed located toward one end where a lid may locate to close the vessel cavity, may include removable fixed bed portions that can be used as samples of such fixed bed. The removable portions may form a part (e.g., a cylindrical “plug”) of the fixed bed attached to a holding portion to allow such plug to be removed through a port in the lid.

In some embodiments, the fixed bed can be arranged with multiple pieces at intermediate angles, or even in random arrangements with respect to fluid flow. Preferably, the fixed bed is oriented to provide essentially isotropic flow behavior. The fixed bed of the current disclosure allows for its use in various applications and bioreactor or container designs, while enabling better and more uniform permeability throughout the bioreactor vessel.

In some embodiments, the fixed bed is integrated in a bioreactor. In some embodiments, the fixed bed is inserted into a bioreactor. In some embodiments, the fixed bed is a monolith. In some embodiments one or more monolithic beds are inserted into a bioreactor. In some embodiments the height of the monolithic bed ranges from 1 to 5 cm and the outer diameter ranges from 2 to 15 cm. In some embodiments, the monolithic bed is integrated in a bioreactor comprising cells and the monolithic bed comprises cells adhered thereto.

According to a further aspect of the disclosure, a process controlled by a controller or computer system with a microprocessor may be provided whereby a user provides via an input (mouse, keyboard, etc.) certain requirements or objectives for constructing a bioreactor, a structured monolithic fixed bed, or both together. For example, the requirements or objectives may be one or more of a desired size, volume or shape, a desired cell density per unit of volume, a desired fluid flow rate, a desired cell line, or other process conditions. The system then determines an optimal construction of the bioreactor to achieve the user-defined objectives, and transmits the information to a 3D printer to fabricate automatically the bioreactor or fixed bed according to these objectives. Alternatively, an application can be presented to the user so that he or she may review the proposed fixed bed design and adjust parameters as needed before finalizing and transmitting such to the printer.

The three-dimensional structure of the fixed bed forming the cell culture bed is advantageous as it may provide a large surface area for culturing adherent cells. Further, the ability to tailor the fixed bed provides for a consistent, repeatable and predictable cell culture, and allows for a desired flow pattern (whether regular or variable) to be created. This includes, for example providing different zones of compaction or density to potentially create a desired flow pattern within the bed to optimize performance in terms of cell growth and viability.

### BRIEF DESCRIPTION OF THE DRAWING FIGURES

FIG. 1 is a perspective view of an exemplary bioreactor for which certain aspects of this disclosure may have applicability;

FIG. 2 is a perspective view of a structured monolithic fixed bed;

FIG. 2A is a cross-sectional view of FIG. 2;

FIG. 2B is a cross-sectional view of an alternative embodiment of a structured monolithic fixed bed;

FIG. 3 is a perspective view of another embodiment of a structured monolithic fixed bed;

FIG. 3A is a cross-sectional view of FIG. 3;

FIG. 4 is a perspective view of another embodiment of a structured monolithic fixed bed;

FIG. 4A is a cross-sectional view of FIG. 4;

FIG. 5 is a perspective view of another embodiment of a structured monolithic fixed bed;

FIG. 5A is a cross-sectional view of FIG. 5;

FIGS. 6, 7, and 8 are schematic views of a structured monolithic fixed bed having differential compaction;

FIG. 9 is a cross-sectional view of an alternative embodiment of a structured monolithic fixed bed;

FIG. 10 is a cross-sectional view of another alternative embodiment of a structured monolithic fixed bed;

FIGS. 11 and 12 illustrate one manner of providing compression to a flexible or compressible structured monolithic fixed bed in a bioreactor;

FIGS. 13, 14, 15, 16, 17 and 18 illustrate various alternative forms of bioreactors; and

FIGS. 19 and 20 illustrate a system and a flow diagram as one example of a technique for forming a custom fixed bed, bioreactor, or both, according to the disclosure.

### DETAILED DESCRIPTION

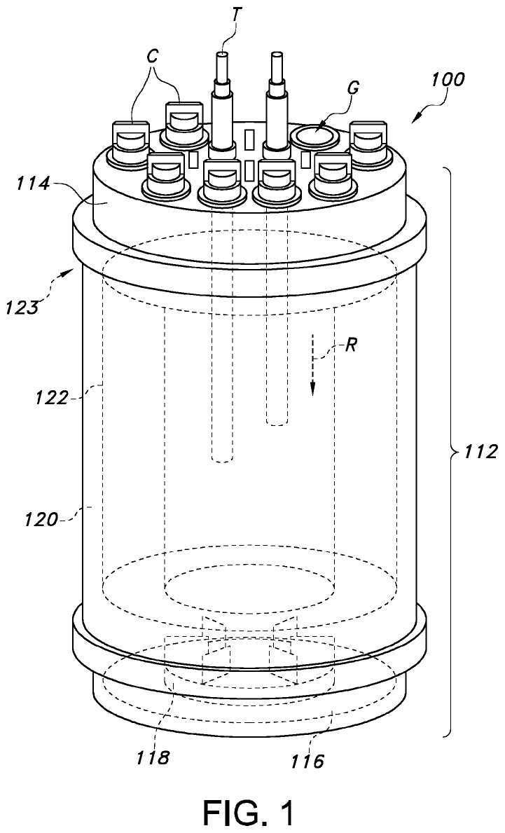

Reference is now made to FIG. 1, which illustrates one embodiment of a fixed bed bioreactor 100 for culturing cells, according to one aspect of the disclosure. In the illustrated example, the bioreactor 100 comprises a vessel formed in part by an external casing or housing 112 forming or including an interior compartment. A cover 114 may be placed on top of the housing 112 to cover or seal the interior compartment, and may be fixed in place or removable. The cover 114 may include various ports or openings G with removable closures or caps C for allowing for the selective introduction or removal of material, fluid, gas, probes, sensors, samplers, or the like, and may also include one or more sensors.

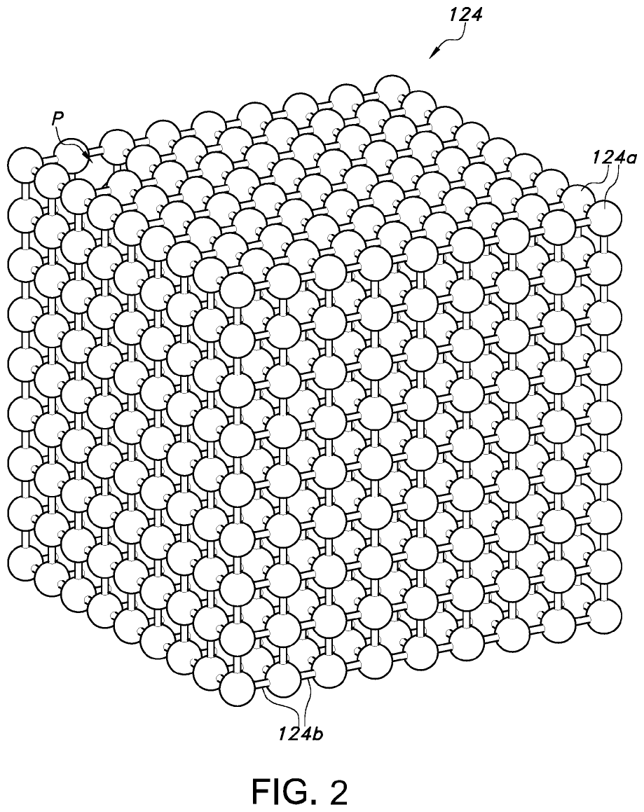

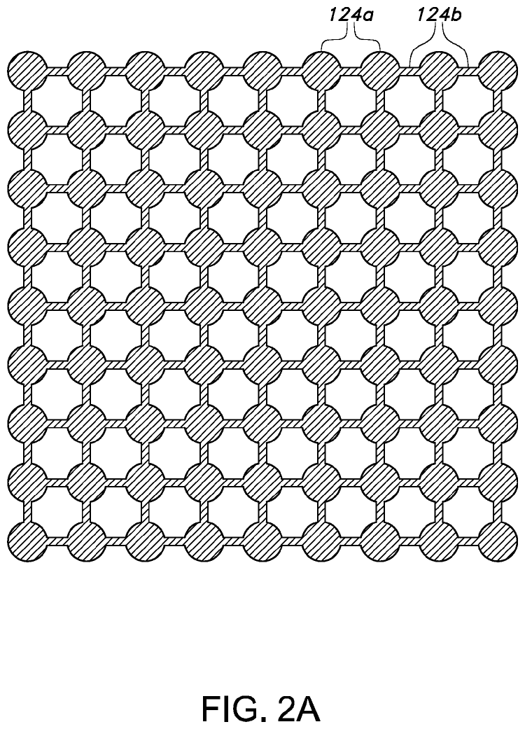

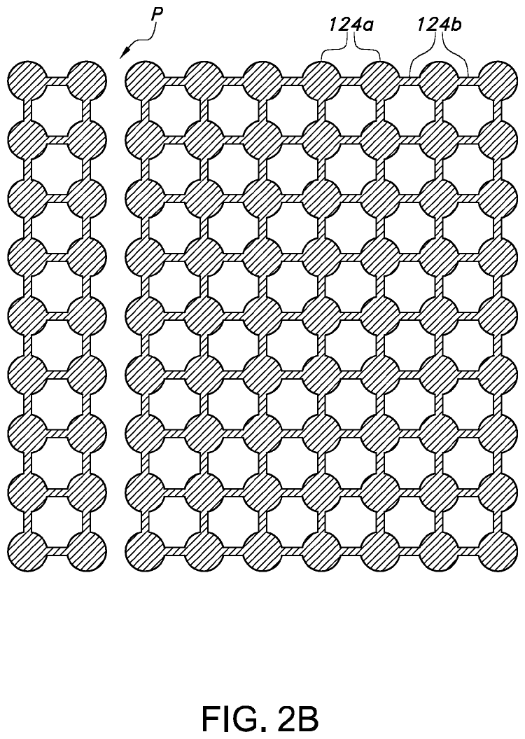

Within the interior compartment of the bioreactor housing 112, several compartments or chambers may be provided for transmitting a flow of liquid, gas, or both, throughout the bioreactor 100. In some embodiments, the chambers may include a first chamber 116 at or near a base of the bioreactor 100. In some embodiments, the first chamber 116 may optionally include an agitator for causing liquid flow within the bioreactor 100. The agitator may be in the form of a rotatable, non-contact magnetic impeller 118, which thus forms a centrifugal pump in the bioreactor. The agitator could also be in the form of an impeller with a mechanical coupling to the base (e.g., via a bearing), with a contact or non-contact drive, or perhaps even an external pump forming part of a liquid circulation system, or any other device for causing liquid circulation within the bioreactor, or perhaps a pump arranged internal or external to the housing 112. As a result of the agitation action provided by the agitator (impeller 118), a fluid such as a liquid may then flow upwardly (as indicated by arrows A in FIG. 2) into a chamber 120 along the outer or peripheral portion of the bioreactor 100 (or otherwise through the fixed bed).

Liquid exiting the chamber 120 is passed to a “headspace” formed in a chamber 123 between one (upper) side of the bed 122 and the cover 114, where the liquid (media) is exposed to a gas (such as oxygen). In some embodiments, liquid may then flow radially inwardly to a central chamber 126 to return to the lower portion of bed 122. In some embodiments, this central chamber 126 can be columnar in nature and may be formed by an imperforate conduit or tube 128 or formed by a central opening or pathway through the structured bed 122.

The chamber 126 returns the liquid to the first chamber 116 (return arrow R) for recirculation through the bioreactor 100, such that a continuous loop results (“bottom to top” in this version). In some embodiments, a sensor, for example a temperature probe or sensor T may also be provided for sensing the temperature of the liquid flowing or residing in the chamber 126. In some embodiments, additional sensors (such as, for example, pH, oxygen, dissolved oxygen, temperature, cell density, etc.) may also be provided at a location before the liquid enters (or re-enters) the chamber 116, including for example at the exit location, or top, of the fixed bed 122.

FIGS. 2 and 2A show one embodiment of a structured fixed bed 122 in the bioreactor of the present disclosure. In one embodiment, the structured fixed bed 122 comprises a three-dimensional (3D) monolith matrix 124 in the form of a scaffold or lattice formed of multiple interconnected units or objects 124a, which have surfaces for cell adhesion (including possibly binding ligands). Preferably, the matrix includes a tortuous path for fluid and cells to flow therethrough when in use. In some embodiments, the matrix 124 may be in the form of a 3D array, lattice, scaffolding, or sponge. The matrix 124 is preferably single use in nature to avoid the cost and complexities involved in cleaning according to bioprocessing standards.

In one embodiment, the matrix 124 is manufactured using 3-D printing technology. In some embodiments, the matrix 124 is manufactured using selective laser sintering (SLS) 3D printing, but is not limited to the use of this method. SLS uses a high-powered laser to fuse powdered material together into a desired 3D shape.

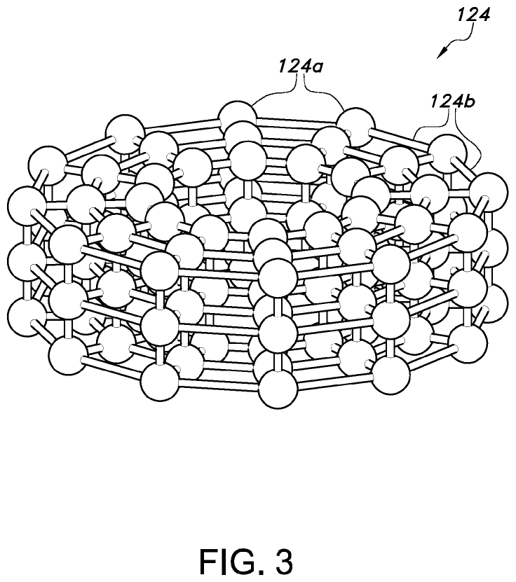

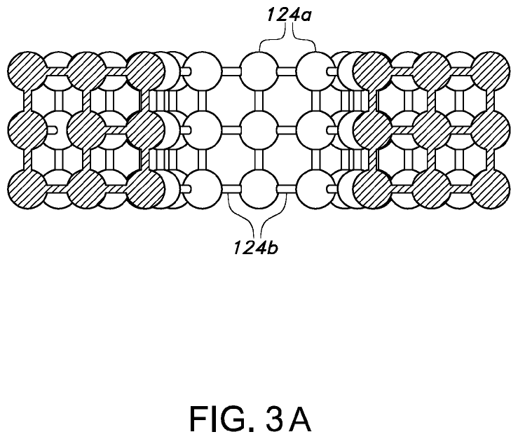

As shown in FIGS. 3 and 3A, the fixed bed 122 may comprise a matrix 124 form of one or more objects 124a that are sintered directly together to form the monolith. The objects 124a may comprise one or more shapes that provide the lowest surface-to-volume ratio for the matrix. The one or more objects 124a may be in the form of beads or spheres, but could be oval, elliptical, cubic hexagonal, octagonal, decahedral, square, rectangular and/or combinations of the foregoing shapes. While other shapes may be used to manufacture the fixed bed 122, pre-positioning of the objects might be desirable to accomplish a homogeneous structure. Thus, a simple sphere or ball may be preferable in such situations due to the lack of any need to maintain the objects in an unstable position before bonding.

The objects 124a are bound or fused together to form the fixed bed structure or matrix, either directly (FIG. 3) or by way of connectors 124b forming spacers. However, the fixed bed 122 could also comprise one or more monolithic sheets of the objects 124a, either connected to each other directly or by connectors 124b. In either case, the structures used may form a tortuous pathway through which fluid may travel in passing through the matrix 124, as outlined further in the following description. The methods of bonding or fusing the objects 124a can include local welding, SLS or other 3D printing methodologies. The surface area of the fixed bed 122 is equal to the surface area of all of the objects 124a and adherent cells would adhere to such objects in a two-dimensional model or manner.

In some embodiments, the objects 124a are solid and manufactured of polymer material. The objects can alternatively be hollow with a cavity inside. The surface of the objects 124a may be continuous, but it may also be discontinuous thereby providing access to the inner cavity inside the object (porosity). If present, the discontinuous portions may form openings that provide access to the cavity and possibly a path for cell culture medium to flow through. The openings may be smaller than the size of an average cell, thereby preventing cells from entering the cavity and getting stuck inside. The opening however can permit cell culture medium to flow therethrough. The fluid flow path through the object may be even or regular, or uneven/irregular.

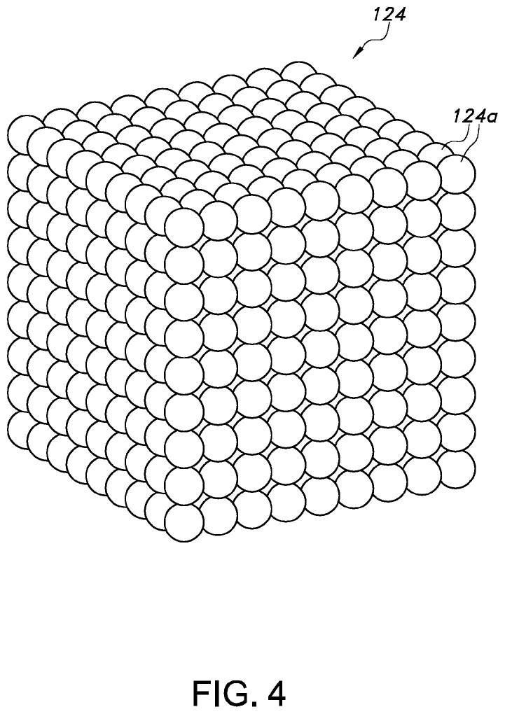

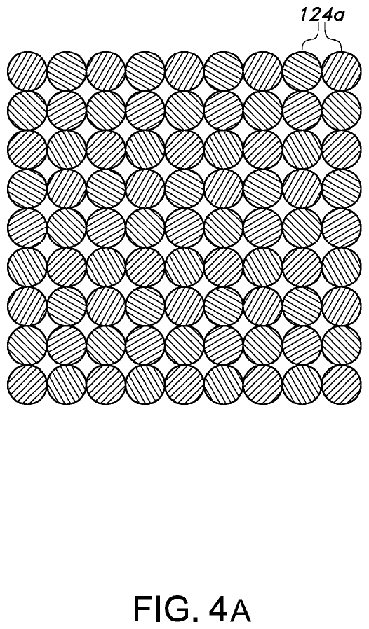

The monolithic matrix 124 may take a variety of shapes. As shown in FIGS. 2 and 3, the monolithic matrix may be a cuboid structure. In the case where the bioreactor 100 includes a differently shaped (e.g., circular, or annular chamber, such as chamber 120, such as in the bioreactor 100 of FIG. 1), a plurality of such structures may be positioned in the chamber, and may be shaped to substantially occupy the space (such as by being wedge-shaped). Alternatively, as shown in FIGS. 4 and 4A, the monolithic matrix 124 may comprise an annular structure.

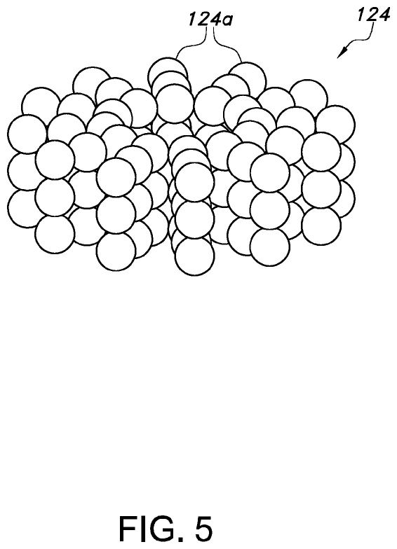

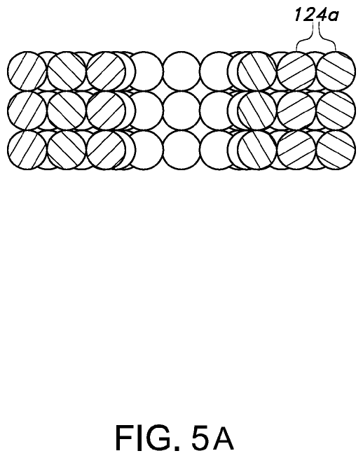

The matrix 124 may be regular, as shown in FIGS. 3 and 4, or irregular. For example, the objects 124a may vary in size, shape, or spacing at various locations along or around the matrix 124. This may allow the matrix 124 to be formed in the desired monolithic manner, but provide variable properties in terms of fluid flow or cell adhesion, depending on the needs of a particular cell culturing operation. As shown in FIGS. 5 and 5A, the objects in the form of beads spheres or other small shapes may also be oriented such that those on an adjacent level contact multiple (e.g., at least two, three or four, five, six, or seven, and in the illustrated example, eight) other objects or beads.

The matrix pathways act to increase fluid flow in both main fluid flow direction and radial diffusion of fluid and cells. Such pathways can have an irregular (e.g., zigzag) or other directional path with components in a main fluid flow direction as well other components with some direction at an angle toward the perpendicular or radial direction. As shown in FIGS. 2 and 2B, the matrix 124 may comprise one or more “chimneys,” pathways P that increase diffusion and decrease pressure drop across the matrix. In some embodiments, the diameter of the pathway(s) P is between 1 and 2 mm and the distance between pathways may be from 1 to 4 mm. The pathway P may be vertical, as shown, or may be horizontal. When multiple stacked matrices are present, the pathways may be offset to enhance the tortuous nature of the fluid flow.

The pathway P, or chimney, is optional and intended to provide preferential flow of the fluid media with some radial flow to distribute cells and media to feed such cells. The need for such pathways may be dependent on the dimensions of the matrix with greater lengths/heights calling for channels.

In some embodiments, the matrix 124 and the bioreactor 100 (in whole or in part) are both manufactured using the same 3D printer or 3D printing process. Forming the unitary matrix 124 and bioreactor 100 in such a manner avoids leakage and the need for seals, provides for simpler manufacturing processes, and ensures the right tolerances exist between the matrix and the bioreactor 100. Sections of the matrix 124 and bioreactor 100 may also be 3D printed, such as for example horizontal slices, and the stacked or assembled together.

In some embodiments, certain portions of the matrix 124, such as located toward one end of the lid 114 may include removable matrix portions that can be used as samples of such matrix. These portions may be shaped to form a part, e.g., a cylindrical “plug”, of the matrix 124 attached to a holding portion to allow such plug to be removed through a port in the lid.

In some embodiments, the matrix 124 can be arranged with multiple pieces at intermediate angles, or even in random arrangements with respect to fluid flow. The matrix 124 may be oriented to provide essentially isotropic flow behavior, which means flow that is invariant with respect to direction. The disclosed matrix 124 may be used in various applications and bioreactor or container designs while enabling better and more uniform permeability throughout the bioreactor vessel.

In some embodiments, the matrix 124 is integrated in a bioreactor 100. In some embodiments, the matrix 124 is inserted into the bioreactor 100. In some embodiments, one or more monoliths, such as the matrix of FIG. 2 or FIG. 4, are inserted into the bioreactor 100. In some embodiments, the height of the monolith ranges from 1 to 5 cm and the outer diameter ranges from 2 to 15 cm. In some embodiments the monolithic matrix 124 is integrated in a bioreactor 100 comprising cells and the monolith comprises cells adhered thereto.

It is believed that the three-dimensional structure of the matrix 124 is advantageous as it provides a large surface area for culturing adherent cells. Further, the matrix 124 may comprise a uniform structure and provide rigidity that enables uniform fluid flow and a consistent and predictable cell culture.

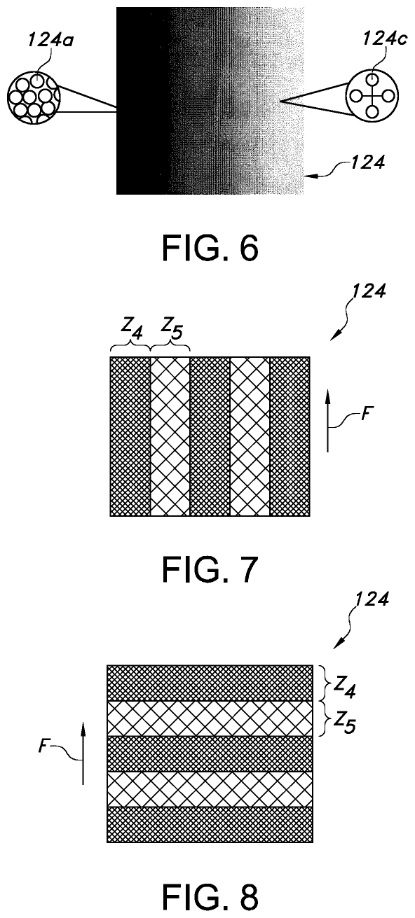

While the above-referenced figures generally show somewhat homogenous arrangements of a matrix, the matrix may be provided in a non-homogenous or non-uniform manner. For example, as illustrated schematically in FIG. 6, the matrix 124 may be provided so as to have a gradient of density or compaction, as indicated by the darker portion to the right and the progressively lighter portion to the left. This may be achieved, for example, by providing objects 124a having a greater degree of compaction in one region or zone Z1, objects 124c of a lesser degree of compaction in a second region or zone Z2, and possibly one or more additional zones Z3 . . . Zn of varying degrees of intermediate compaction therebetween. By using additive manufacturing, such as 3D printing techniques, the different degrees of compaction may be achieved using objects 124a, 124b of different sizes, different shapes, different spacing, or any combination of the foregoing, and thus provide the matrix 124 with variable or characteristics in terms of porosity and hence fluid flow. In this way, it is possible for a structured monolithic matrix 124 to simulate the effect of multiple layers in a conventional fixed bed formed of woven or non-woven materials, when in fact no discrete layers are present.

Turning to FIGS. 7 and 8, it can be understood that the structured monolithic matrix 124 may include two or more different zones of differing or alternating compaction or density. For example, the arrangement in FIG. 7 shows three zones Z4 of high density or compaction, each separated by a zone Z5 of low density or compaction. The zones Z5 of low density or compaction may thus function essentially as spacers for the higher compaction zones Z4, which can be used primarily for adherent cell growth. The lower compaction zones or regions (high porosity) thus have greater porosity and promote fluid flow through the matrix 124 and between adjacent higher compaction (low porosity) regions or zones. Any number of such zones may be provided in any desired pattern or arrangement, and each zone or region provided may have varying compaction in any direction. Furthermore, while FIG. 7 shows the zones as being vertically arranged, it can be understood from FIG. 8 that the zones Z4, Z5 may be horizontally arranged (with a primarily flow direction F being vertical, but generally horizontal flow is also possible, as outlined further in the following description).

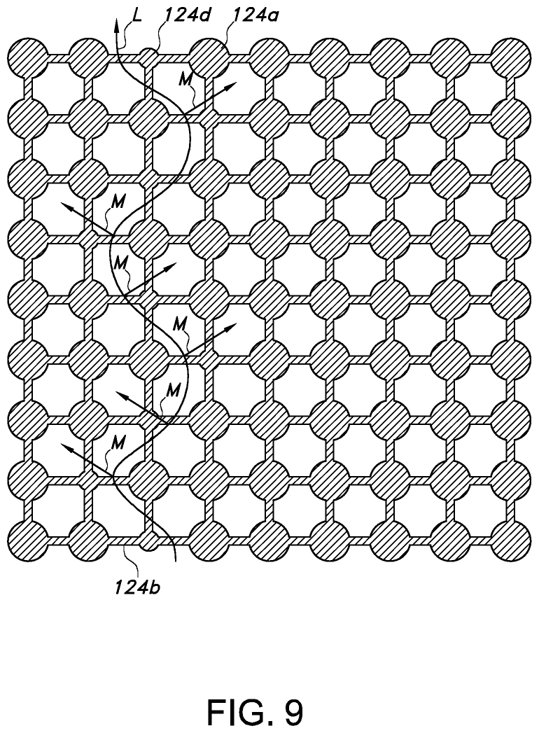

While the zones of differential compaction are shown above as being linear, the arrangement of the matrix 124 formed using additive manufacturing techniques may be such that a zone of low compaction creates a labyrinth pathway L, as shown in FIG. 9. In one example, this may be achieved by having objects 124d of a first smaller size or greater spacing provided along the desired pathway, bounded by objects 124a of a greater size or lesser spacing, as shown. The objects 124a, 124d may optionally be connected by connectors 124b, which may also be of different sizes and shapes. The arrangement may be such that fluid flow in different directions (so called “off ramps” creating convection currents throughout the matrix 124, as indicated by arrows M) into the higher compaction areas is achieved. Although only one such labyrinth pathway L is shown in FIG. 9, it should be appreciated that more than one such pathway may be provided, and further that the pathway may extend in any direction.

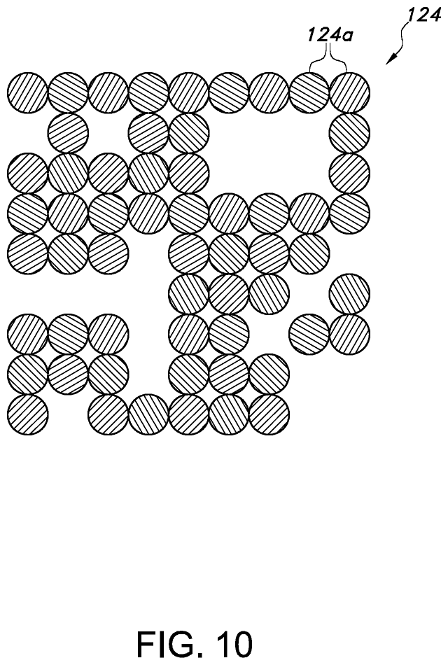

In the previous examples, the objects 124a, 124c, 124d are illustrated as generally being orderly and thus having a regular pattern. However, it is possible for the objects forming form the 3D printed monolithic matrix 124 to be arranged randomly, or having an irregular pattern with different structure sizes (for example, the arrangement could accommodate large channels, smaller ones and cavities for the cells). FIG. 10 illustrates one example of a random arrangement of similarly sized objects 124a, but the randomness could also apply to the size, shape, or spacing of the objects forming the 3D printed structured monolithic matrix 124. The bioreactor could be composed of several sub modules and then include several monoliths.

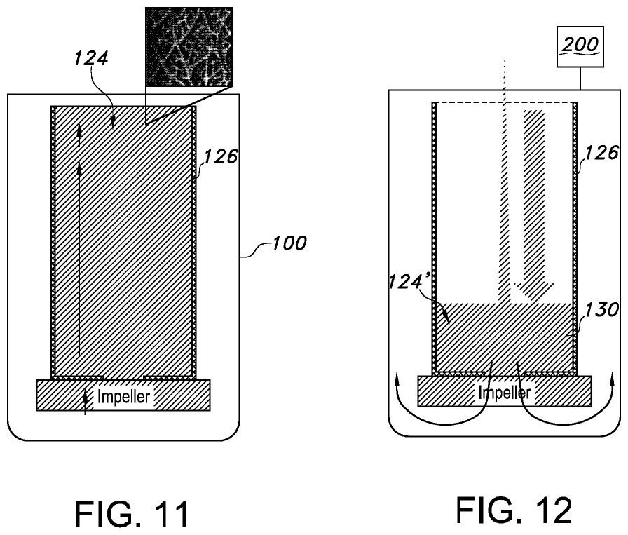

The materials used to form the matrix 124 are biocompatible, and may be rigid or flexible. In the case of flexible materials, the matrix 124 may be compressible, and thus essentially function like a sponge. Thus, as shown in FIGS. 11 and 12, by compressing the matrix 124 (which in the enlarged view is shown as being formed of a random web of 3D printed material) within an interior central compartment or chamber 126 of a bioreactor 100, fluid may be released therefrom (and possibly transferred to a reservoir 200 in fluid communication with the bioreactor, such as a harvest bottle connected by a drain line). This compression may be achieved in a variety of ways, such as by using a compressor in the form of a movable plunger 130, or engaging the matrix 124 using a movable internal or external wall also forming a compressor or plunger. Cell detachment from the matrix 124 may be achieved such as by using Trypsin or similar detachment agents, either prior to or during the compression of the matrix 124. The compression of the matrix 124 may be reversed to allow for expansion, and then repeated to compress the matrix and facilitate dispersing the cell detachment agent therethrough. Additionally or alternatively, a vacuum may be applied to the bioreactor 100 or any portion thereof (e.g., the central chamber 126) to aid in the fluid recovery.

During the additive manufacturing (e.g., 3D printing) process, the matrix 124 may be formed using a single material or a variety of materials, such as by using plural devices, such as for example 3D printing heads, nozzles, or extruders concurrently or sequentially. In one example, the matrix 124 may be formed of both a soluble and an insoluble material, and then a solvent (e.g., water) applied to wash away the soluble material, which may be used to form the desired patterns within the matrix. This technique could be used, for example, to achieve the random configuration example shown in FIG. 10.

Various treatments may also be applied to the materials to provide certain qualities, such as by making certain portions of the bed hydrophilic and certain portions hydrophobic, for example. Likewise, certain portions may be made cell adherent, such as for example by providing binding ligands. Any or all such treatments may also be applied to the material(s) once the matrix 124 is fabricated, during the forming process, or both.

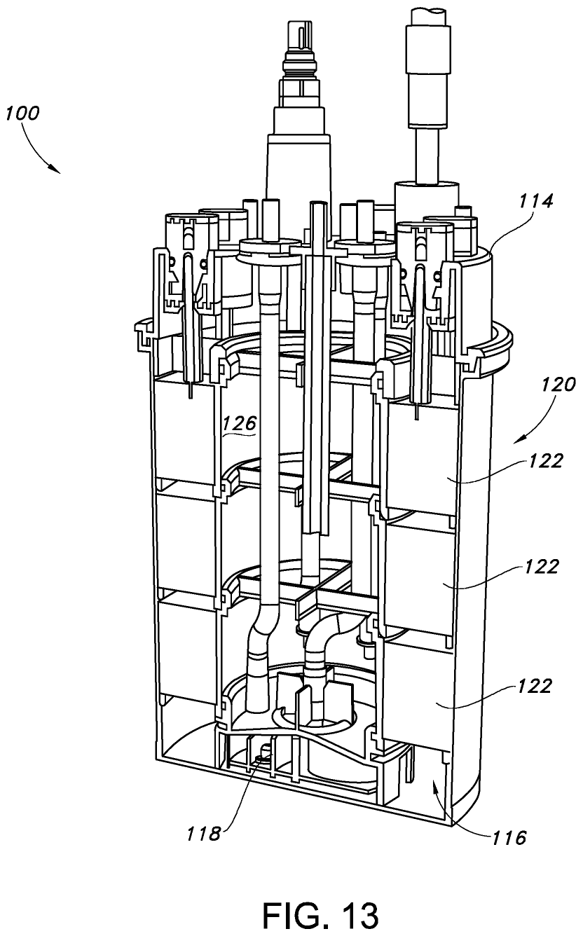

FIG. 13 illustrates a further example of a bioreactor 100. This bioreactor 100 includes the basic structure of the embodiment of FIG. 1, but with multiple stacked beds 122. Each bed 122 may be formed by one or more matrixes (two shown, but any number could be provided).

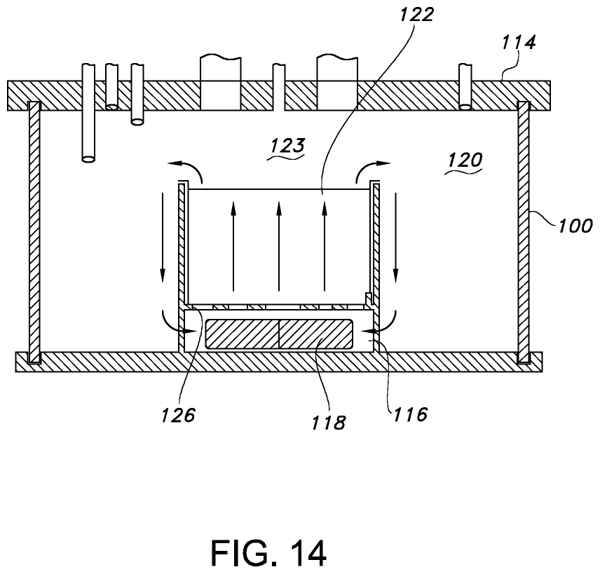

As shown in FIG. 14, the bed 122 may be located in the radially inward (central) chamber 126. The impeller 118 may be in a base chamber 116. The impeller 118 when activated may cause fluid, such as liquid, gas, or both, to flow upwardly through the bed 122, outwardly to chambers 120, 123, and then back to the base chamber 116.

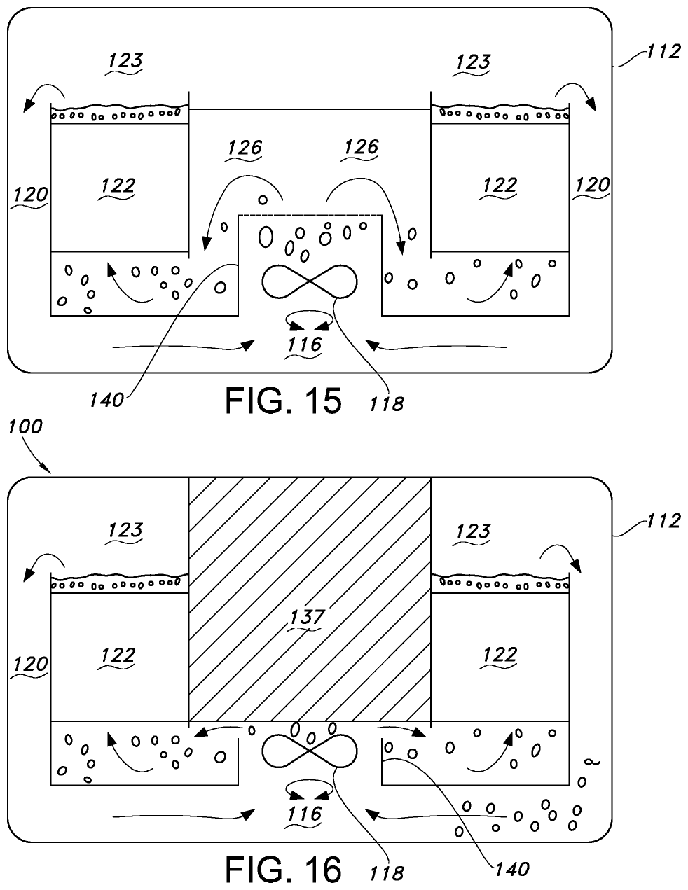

Turning to FIG. 15, an alternative embodiment of a bioreactor 100 is illustrated, which as above comprises a housing 112. In this embodiment, fluid is circulated by an agitator such as an impeller 118 in a housing or container 140 in or near the base chamber 116 and flows to a central chamber 126 first, then radially outwardly to pass vertically through a structured fixed bed 122. The liquid upon exiting the upper portion of the bed 122 then returns along a chamber 120 radially outward of the bed 122, and is returned to the impeller 118 for repeating the cycle.

FIG. 16 illustrates an alternative embodiment of a bioreactor 100 somewhat similar in construction to that of FIG. 15, with the main exception that the central chamber is omitted takes the form of a solid core 137. Thus, fluid, such as liquid, may exit the container 140 for impeller 118 radially through openings, and travel essentially as previously described, returning to the container 140 via base chamber 116.

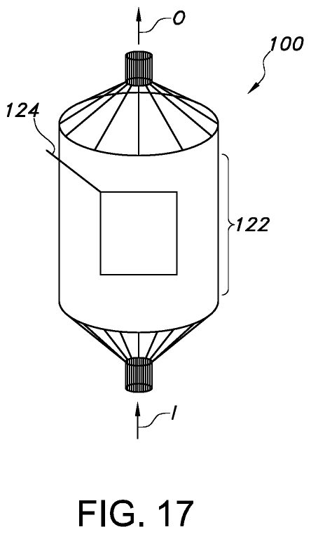

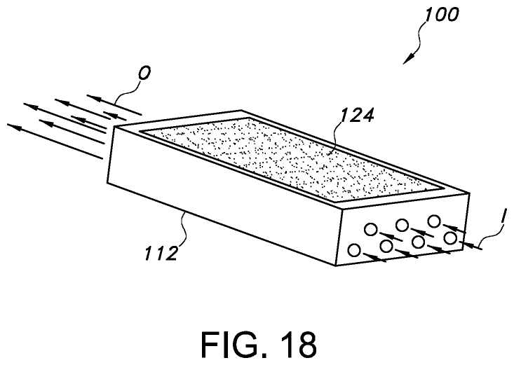

FIG. 17 illustrates an embodiment in which a structured fixed bed 122 is comprised of one or more monolithic matrices 124 positioned in a bioreactor 100. The bioreactor is arranged such that flow passes, or perfuses, from an inlet I to an outlet O, such as vertically from top to bottom, but could be reversed in direction. FIG. 18 shows a further arrangement of a bioreactor 100 where the flow passes generally horizontally from an inlet I to an outlet O through a structured fixed bed 122 comprised of one or more monolithic matrices 124 positioned in a bioreactor 100 in a stacked arrangement.

As noted above, the bioreactor 100 and matrix 124 may together be concurrently formed as a unitary structure during a single additive manufacturing (e.g., 3D printing) process. This advantageously avoids the need for separately constructing and installing the matrix 124 in the bioreactor with the necessary seals being provided by adhesives or otherwise. The result is a bioreactor with greater integrity, quality and uniformity/consistency of manufacture and functioning.

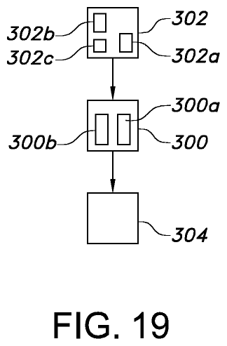

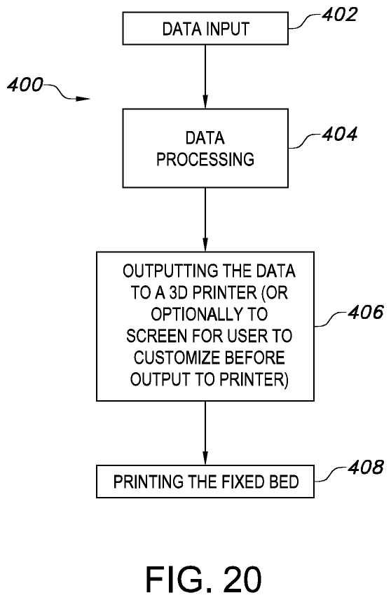

According to a further aspect of the disclosure, and with reference to FIGS. 19 and 20, a process controlled by a computer 300 including a microprocessor 300a and storage 300b with an application stored thereon (or downloaded from the cloud) may be provided. A user may provide via an input 302 (such as a keyboard 302a, mouse 302b, and display 302c) certain objectives for constructing a bioreactor, a structured monolithic bed, or both together. For example, the objectives may be one or more of a desired size, volume or shape, a desired cell density per unit of volume, a desired fluid flow rate, a desired cell line, or other process conditions. The computer 300 then determines an optimal construction of the bioreactor to achieve the user-defined objectives, and outputs the information to an output device, such as a device for performing additive manufacturing (such as a 3D printer 304) to fabricate the bioreactor or matrix according to these objectives, or optionally to the display 302c for user customization before output to the printing device. While discrete units are shown, it should be appreciated that all could be combined into a single device.

FIG. 20 illustrates via flowchart a corresponding process 400 for forming a structured monolithic fixed bed. The method includes a data input step 402, a data processing step 404, the step 406 of outputting the data to a 3D printer or optionally to screen for user to customize before output to printer, and the step 408 of printing a fixed bed with the printer based on final inputs and application.

Summarizing, this disclosure may relate to one or more of the following items in any ordered combination:
1. An apparatus for culturing cells, comprising:
   a bioreactor vessel; and
   a monolithic structured cell culture bed disposed in a portion of the vessel.
2. The apparatus of item 1, further including an agitator for flowing fluid through the monolithic structured cell culture bed.
3. The apparatus of item 1 or item 2, wherein the monolithic structured cell culture bed is annular.
4. The apparatus of any of items 1-3, wherein the monolithic structured cell culture bed is cuboid.
5. The apparatus of any of items 1-4, wherein the monolithic structured cell culture bed comprises a sheet of interconnected objects having a partially curved or rounded shape.
6. The apparatus of any of items 1-5, wherein the monolithic structured cell culture bed comprises a three dimensional matrix of objects.
7. The apparatus of item 6, wherein the objects in the matrix are directly connected.
8. The apparatus of item 6, wherein the objects in the matrix are connected by connectors forming a space between the objects.
9. The apparatus of any of items 1-9, wherein the bioreactor vessel includes an annular chamber for receiving the monolithic structured cell culture bed.
10. The apparatus of any of items 1-9, wherein the bioreactor vessel and the monolithic structured cell culture bed comprise a unitary structure.
11. The apparatus of any of items 1-10, wherein the monolithic structured cell culture bed comprises one or more pathways for unobstructed fluid flow.
12. The apparatus of item 11, wherein the one or more pathways are linear.
13. The apparatus of item 11, wherein the one or more pathways are non-linear.
14. The apparatus of any of items 1-13, wherein the monolithic structured cell culture bed comprises randomly arranged objects.
15. The apparatus of any of items 1-14, wherein the monolithic structured cell culture bed comprises a first zone having a greater density of objects than a second zone.
16. The apparatus of any of items 1-15, wherein the monolithic structured cell culture bed is adapted to create a fluid flow gradient.
17. The apparatus of any of items 1-16, wherein the monolithic structured cell culture bed is 3D printed.
18. The apparatus of any of items 1-17, wherein the monolithic structured cell culture bed is compressible.
19. The apparatus of item 18, further including a compressor for compressing the monolithic structured cell culture bed.
20. The apparatus of any of items 1-19, wherein the monolithic structured cell culture bed includes regions of varying porosity.
21. The apparatus according to item 20, wherein the regions include a larger porosity region for effecting culture medium distribution and a smaller porosity region for cell entrapment/growth.
22. The apparatus according to any of items 1-21, wherein the monolithic structured cell culture bed includes at least a portion treated to be hydrophilic.
23. The apparatus according to any of items 1-22, wherein the monolithic structured cell culture bed includes at least a portion treated to be hydrophobic.
24. The apparatus according to any of items 1-23, wherein the monolithic structured cell culture bed includes at least a portion treated for increased cell adhesion characteristics.
25. A method for manufacturing, comprising:
   forming via additive manufacturing a monolithic structured cell culture bed.
26. The method of item 25, wherein the forming step comprises 3D printing the monolithic structured cell culture bed.
27. The method of item 25 or item 26, comprising forming a bioreactor and the monolithic structured cell culture bed together as a unitary structure.
28. The method of any of items 25-27, wherein the forming step comprises 3D printing the bioreactor and the monolithic structured cell culture bed concurrently.
29. The method of any of items 25-28, wherein the forming step comprises forming the monolithic structured cell culture bed using two different materials.
30. The method of any of items 25-29, further including the step of functionally modifying at least a portion of the monolithic structured cell culture bed during or after the forming step.
31. The method of any of items 25-30, further including the steps of:
   inputting one or more desired objectives for the monolithic structured cell culture bed into a computer, and controlling the forming step using the computer based on the one or more desired objectives.
32. An apparatus for culturing cells, comprising:
   a monolithic structured cell culture bed including one or more pathways within the monolithic structured cell culture bed for promoting unobstructed fluid flow.
33. The apparatus of item 32, wherein the one or more pathways are linear.
34. The apparatus of item 32, wherein the one or more pathways are non-linear.
35. An apparatus for culturing cells, comprising:
   a monolithic structured cell culture bed comprising randomly arranged objects.
36. An apparatus for culturing cells, comprising:
   a monolithic structured cell culture bed comprising a first zone having a greater density of objects than a second zone.
37. An apparatus for culturing cells, comprising:
   a monolithic structured cell culture bed adapted to create a fluid flow gradient.
38. An apparatus for culturing cells, comprising:
   a compressible monolithic structured cell culture bed.
39. The apparatus of item 38, further including a compressor for compressing the monolithic structured cell culture bed.
40. An apparatus for culturing cells, comprising:
   a monolithic structured cell culture bed comprising regions of varying porosity.
41. The apparatus according to item 40, wherein the regions include a larger porosity region for effecting culture medium distribution and a smaller porosity region for cell entrapment/growth.
42. An apparatus for culturing cells, comprising:
   a monolithic structured cell culture bed comprising at least a portion treated so as to be hydrophilic.
43. An apparatus for culturing cells, comprising:
   a monolithic structured cell culture bed comprising at least a portion treated so as to be hydrophobic.
44. An apparatus for culturing cells, comprising:
   a monolithic structured cell culture bed comprising at least a portion treated for increased cell adhesion characteristics.
45. The apparatus according to item 44, wherein the portion treated for increased cell adhesion characteristics comprises binding ligands.
46. A bioreactor including the apparatus of any of items 32-45.
47. A bioreactor including the apparatus of any of items 32-45 formed as a unitary structure.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” “generally” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., “component includes” does not exclude or preclude the presence of additional, non-recited components, features, element, members, steps, known in the art or disclosed therein.

“Monolith” or “monolithic” as used herein means a single three-dimensional structure which avoids discrete portions such as elements or layers placed on top of or adjacent one another to form the whole.

While certain embodiments have been shown and described herein, it will be obvious to those skilled in the art that such embodiments are provided by way of example only. Numerous variations, changes, and substitutions will now occur to those skilled in the art without departing from the invention. For example, while the bioreactor is shown in a vertical orientation, it could be used in any orientation. The bioreactor may also be formed of rigid, flexible, or semi-flexible materials, and may be made for single or multiple uses. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1. An apparatus for culturing cells, comprising:
a bioreactor vessel; and
a monolithic structured cell culture bed disposed in a portion of the vessel.

2. The apparatus of claim 1, further including an agitator for flowing fluid through the monolithic structured cell culture bed.

3. The apparatus of claim 1, wherein the monolithic structured cell culture bed is annular.

4. The apparatus of claim 1, wherein the monolithic structured cell culture bed is cuboid.

5. The apparatus of claim 1, wherein the monolithic structured cell culture bed comprises a sheet of interconnected objects having a partially curved or rounded shape.

6. The apparatus of claim 1, wherein the monolithic structured cell culture bed comprises a three-dimensional matrix of objects.

7. The apparatus of claim 6, wherein the objects in the three-dimensional matrix are directly connected.

8. The apparatus of claim 6, wherein the objects in the three-dimensional matrix are connected by connectors forming a space between the objects.

9. The apparatus of claim 1, wherein the bioreactor vessel includes an annular chamber for receiving the monolithic structured cell culture bed.

10. The apparatus of claim 1, wherein the bioreactor vessel and the monolithic structured cell culture bed comprise a unitary structure.

11. The apparatus of claim 1, wherein the monolithic structured cell culture bed comprises one or more pathways for unobstructed fluid flow.

12. The apparatus of claim 11, wherein the one or more pathways are linear.

13. The apparatus of claim 11, wherein the one or more pathways are non-linear.

14. The apparatus of claim 1, wherein the monolithic structured cell culture bed comprises randomly arranged objects.

15. The apparatus of claim 1, wherein the monolithic structured cell culture bed comprises a first zone having a greater density of objects than a second zone.

16. The apparatus of claim 1, wherein the monolithic structured cell culture bed is adapted to create a fluid flow gradient.

17. The apparatus of claim 1, wherein the monolithic structured cell culture bed is 3D printed.

18. The apparatus of claim 1, wherein the monolithic structured cell culture bed is compressible.

19. The apparatus of claim 18, further including a compressor for compressing the monolithic structured cell culture bed.

20. The apparatus of claim 1, wherein the monolithic structured cell culture bed includes regions of varying porosity.
